(19) United States
(12) Patent Application Publication
(10) Pub. No.: US 2005/0165013 A1
(43) Pub. Date: Jul. 28, 2005

(54) Pharmaceutical compositions containing anticholinergics and EGFR kinase inhibitors

(75) Inventors: Christopher J. M. Meade, Maselheim (DE); Michel Pairet, Biberach (DE); Michael P. Pieper, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 11/087,153

(22) Filed: Mar. 23, 2005

Related U.S. Application Data
(63) Continuation of application No. 10/614,382, filed on Jul. 7, 2003.
(60) Provisional application No. 60/407,746, filed on Sep. 3, 2002.

(30) Foreign Application Priority Data
Jul. 9, 2002 (DE) DE 102 30 751.2

(51) Int. Cl. A61K 31/5377; A61K 31/4745; A61K 31/517

(52) U.S. Cl. 514/234.2; 514/291; 514/266.4

(57) ABSTRACT

The present invention relates to novel pharmaceutical compositions based on new anticholinergics and EGFR kinase inhibitors, processes for preparing them and their use in the treatment of respiratory diseases.

### RELATED APPLICATIONS

This application is a continuation of U.S. Ser. No. 10/614,382, filed on Jul. 7, 2003, now abandoned, which claimed benefit of U.S. Provisional Application Ser. No. 60/407,746, filed on Sep. 3, 2002, each of which is herein incorporated by reference in its entirety.

### FIELD OF THE INVENTION

The present invention relates to novel pharmaceutical compositions based on new anticholinergics and EGFR kinase inhibitors, processes for preparing them and their use in the treatment of respiratory complaints.

### BRIEF DESCRIPTION OF THE DRAWING

FIG. 1 depicts a particularly preferred inhaler for administering the pharmaceutical composition according to the present invention.

### DETAILED DESCRIPTION OF THE INVENTION

Surprisingly, an unexpectedly beneficial therapeutic effect, particularly a synergistic effect can be observed in the treatment of inflammatory and/or obstructive diseases of the respiratory tract if one or more, preferably one, anticholinergic of formula 1 is used with one or more, preferably one, EGFR-kinase inhibitor 2. In view of this synergistic effect, the pharmaceutical combinations according to the invention can be used in smaller doses than would be the case with the individual compounds used in monotherapy in the usual way.

The combinations of active substances according to the invention are surprisingly characterized both by a rapid onset of activity and also by a long-lasting duration of activity. This is very important to the patient's feeling of well-being, as on the one hand they experience a rapid improvement in their condition once the combination has been administered and on the other hand the drug need only be taken once a day, thanks to its long-lasting effects.

These effects are observed both when the active substances are administered simultaneously within a single active substance formulation and also when the two active substances are administered successively in separate formulations. It is preferable according to the invention to administer the two active ingredients simultaneously in a single formulation.

Within the scope of the present invention the anticholinergics used are the salts of formula 1 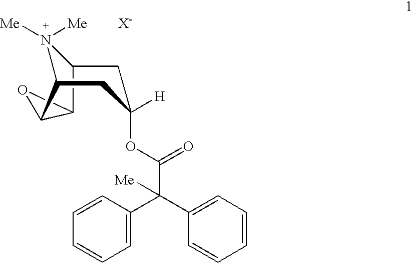
wherein:
X⁻ denotes an anion with a single negative charge, preferably an anion selected from the group consisting of chloride, bromide, iodide, sulfate, phosphate, methanesulfonate, nitrate, maleate, acetate, citrate, fumarate, tartrate, oxalate, succinate, benzoate, and p-toluenesulfonate.

Preferably, the salts of formula 1 are used wherein:
X⁻ denotes an anion with a single negative charge selected from the group consisting of chloride, bromide, 4-toluenesulfonate, and methanesulfonate, preferably bromide.

Most preferably, the salts of formula 1 are used wherein:
X⁻ denotes an anion with a single negative charge selected from the group consisting of chloride, bromide, and methanesulfonate, preferably bromide.

Particularly preferred according to the invention is the salt of formula 1 wherein X⁻ denotes bromide.

The salts of formula I are known from International Patent Application WO 02/32899.

Within the scope of the present patent application, an explicit reference to the pharmacologically active cation of formula 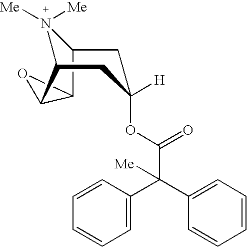
can be recognized by the use of the designation 1′. Any reference to compounds 1 naturally includes a reference to the cation 1′.

Within the scope of the present invention the preferred EGFR kinase inhibitors (2) are compounds which are selected from among: 4-[(3-chloro-4-fluorophenyl)amino]-7-(2-{4-[(S)-(2-oxotetrahydrofuran-5-yl)carbonyl]piperazin-1-yl}ethoxy)-6-[(vinylcarbonyl)amino]quinazoline, 4-[(3-chloro-4-fluorophenyl)amino]-7-[2-((S)-6-methyl-2-oxomorpholin-4-yl)ethoxy]-6-[(vinylcarbonyl)amino]quinazoline, 4-[(3-chloro-4-fluorophenyl)amino]-7-[4-((R)-6-methyl-2-oxomorpholin-4-yl)butyloxy]-6-[(vinylcarbonyl)amino]quinazoline, 4-[(3-chloro-4-fluorophenyl)amino]-7-[4-((S)-6-methyl-2-oxomorpholin-4-yl)butyloxy]-6-[(vinylcarbonyl)amino]quinazoline, 4-[(3-chloro-4-fluorophenyl)amino]-6-{[4-(morpholin-4-yl)-1-oxo-2-buten-1-yl]amino}-7-cyclopropylmethoxyquinazoline, 4-[(3-chloro-4-fluorophenyl)amino]-6-{[4-(N,N-diethylamino)-1-oxo-2-buten-1-yl]amino}-7-cyclopropylmethoxyquinazoline, 4-[(3-chloro-4-fluorophenyl)amino]-6-{[4-(N,N-dimethylamino)-1-oxo-2-buten-1-yl]amino}-7-cyclopropylmethoxyquinazoline, 4-[(3-chloro-4-fluorophenyl)amino]-6-[(4-{N-[2-(ethoxycarbonyl)ethyl]-N-[(ethoxycarbonyl)methyl]amino}-1-oxo-2-buten-1-yl)amino]-7-cyclopropylmethoxyquinazoline, 4-[(R)-(1-phenylethyl)amino]-6-{[4-(morpholin-4-yl)-1-oxo-2-buten-1-yl]amino}-7-cyclopropylmethoxyquinazoline, 4-[(R)-(1-phenylethyl)amino]-6-{[4-(morpholin-4-yl)-1-oxo-2-buten-1-yl]amino}-7-cyclopentyloxyquinazoline, 4-[(3-chloro-4-fluorophenyl)amino]-6-{[4-((R)-6-methyl-2-oxomorpholin-4-yl)-1-oxo-2-buten-1-yl]amino}-7-cyclopropylmethoxyquinazoline, 4-[(3-chloro-4-fluorophenyl)amino]-6-{[4-((R)-6-methyl-2-oxomorpholin-4-yl)-1-oxo-2-buten-1-yl]amino}-7-[(S)-(tetrahydrofuran-3-yl)oxy]quinazoline, 4-[(3-chloro-4-fluorophenyl)amino]-6-{[4-((R)-2-methoxymethyl-6-oxomorpholin-4-yl)-1-oxo-2-buten-1-yl]amino}-7-cyclopropylmethoxyquinazoline, 4-[(3-chloro-4-fluorophenyl)amino]-6-[2-((S)-6-methyl-2-oxomorpholin-4-yl)ethoxy]-7-methoxyquinazoline, 4-[(3-chloro-4-fluorophenyl)amino]-6-({4-[N-(2-methoxyethyl)-N-methylamino]-1-oxo-2-buten-1-yl}amino)-7-cyclopropylmethoxyquinazoline, 4-[(3-chloro-4-fluorophenyl)amino]-6-{[4-(N,N-dimethylamino)-1-oxo-2-buten-1-yl]amino}-7-cyclopentyloxyquinazoline, 4-[(R)-(1-phenylethyl)amino]-6-{[4-(N,N-bis-(2-methoxyethyl)amino)-1-oxo-2-buten-1-yl]amino}-7-cyclopropylmethoxyquinazoline, 4-[(R)-(1-phenylethyl)amino]-6-({4-[N-(2-methoxyethyl)-N-ethylamino]-1-oxo-2-buten-1-yl}amino)-7-cyclopropylmethoxyquinazoline, 4-[(R)-(1-phenylethyl)amino]-6-({4-[N-(2-methoxyethyl)-N-methylamino]-1-oxo-2-buten-1-yl}amino)-7-cyclopropylmethoxyquinazoline, 4-[(R)-(1-phenylethyl)amino]-6-({4-[N-(tetrahydropyran-4-yl)-N-methylamino]-1-oxo-2-buten-1-yl}amino)-7-cyclopropylmethoxyquinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)quinazoline, 4-[(3-chloro-4-fluorophenyl)amino]-6-({4-[N-(2-methoxyethyl)-N-methylamino]-1-oxo-2-buten-1-yl}amino)-7-cyclopentyloxyquinazoline, 4-[(3-chloro-4-fluorophenyl)amino]-6-{[4-(N-cyclopropyl-N-methylamino)-1-oxo-2-buten-1-yl]amino}-7-cyclopentyloxyquinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]quinazoline, 4-[(3-chloro-4-fluorophenyl)amino]-6-[3-(morpholin-4-yl)propyloxy]-7-methoxyquinazoline, 4-[(3-ethynylphenyl)amino]-6,7-bis-(2-methoxyethoxy)quinazoline, 4-[(3-chloro-4-fluorophenyl)amino]-7-[3-(morpholin-4-yl)propyloxy]-6-[(vinyl-carbonyl)amino]quinazoline, 4-[(R)-(1-phenylethyl)amino]-6-(4-hydroxyphenyl)-7H-pyrrolo[2,3-d]pyrimidin, 3-cyano-4-[(3-chloro-4-fluorophenyl)amino]-6-{[4-(N,N-dimethylamino)-1-oxo-2-buten-1-yl]amino}-7-ethoxyquinoline, 4-{[3-chloro-4-(3-fluorobenzyloxy)phenyl]amino}-6-(5-{[(2-methansulfonylethyl)amino]methyl}furan-2-yl)quinazoline, Cetuximab, Trastuzumab, ABX-EGF, and Mab ICR-62.

More preferred EGFR kinase inhibitors 2 are selected from among the group consisting of 4-[(3-chloro-4-fluorophenyl)amino]-7-(2-{4-[(S)-(2-oxotetrahydrofuran-5-yl)-carbonyl]piperazin-1-yl}ethoxy)-6-[(vinylcarbonyl)amino]quinazoline, 4-[(3-chloro-4-fluorophenyl)amino]-7-[2-((S)-6-methyl-2-oxomorpholin-4-yl)ethoxy]-6-[(vinylcarbonyl)amino]quinazoline, 4-[(3-chloro-4-fluorophenyl)amino]-7-[4-((R)-6-methyl-2-oxomorpholin-4-yl)butyloxy]-6-[(vinylcarbonyl)amino]quinazoline, 4-[(3-chloro-4-fluorophenyl)amino]-7-[4-((S)-6-methyl-2-oxomorpholin-4-yl)butyloxy]-6-[(vinylcarbonyl)amino]quinazoline, 4-[(3-chloro-4-fluorophenyl)amino]-7-[4-(2,2-dimethyl-6-oxomorpholin-4-yl)butyloxy]-6-[(vinylcarbonyl)amino]quinazoline, 4-[(3-chloro-4-fluorophenyl)amino]-6-{[4-(morpholin-4-yl)-1-oxo-2-buten-1-yl]amino}-7-cyclopropylmethoxyquinazoline, 4-[(3-chloro-4-fluorophenyl)amino]-6-{[4-(N,N-diethylamino)-1-oxo-2-buten-1-yl]amino}-7-cyclopropylmethoxyquinazoline, 4-[(3-chloro-4-fluorophenyl)amino]-6-{[4-(N,N-dimethylamino)-1-oxo-2-buten-1-yl]amino}-7-cyclopropylmethoxyquinazoline, 4-[(3-chloro-4-fluorophenyl)amino]-6-[(4-{N-[2-(ethoxycarbonyl)ethyl]-N-[(ethoxycarbonyl)methyl]amino}-1-oxo-2-buten-1-yl)amino]-7-cyclopropylmethoxyquinazoline, 4-[(R)-(1-phenylethyl)amino]-6-{[4-(morpholin-4-yl)-1-oxo-2-buten-1-yl]amino}-7-cyclopropylmethoxyquinazoline, 4-[(R)-(1-phenylethyl)amino]-6-{[4-(morpholin-4-yl)-1-oxo-2-buten-1-yl]amino}-7-cyclopentyloxyquinazoline, 4-[(3-chloro-4-fluorophenyl)amino]-6-{[4-((R)-6-methyl-2-oxomorpholin-4-yl)-1-oxo-2-buten-1-yl]amino}-7-cyclopropylmethoxyquinazoline, 4-[(3-chloro-4-fluorophenyl)amino]-6-({4-[bis-(2-methoxyethyl)amino]-1-oxo-2-buten-1-yl}amino)-7-cyclopropylmethoxyquinazoline, 4-[(3-chloro-4-fluorophenyl)amino]-6-{[4-((R)-6-methyl-2-oxomorpholin-4-yl)-1-oxo-2-buten-1-yl]amino}-7-[(S)-(tetrahydrofuran-3-yl)oxy]quinazoline, 4-[(3-chloro-4-fluorophenyl)amino]-6-{[4-((R)-2-methoxymethyl-6-oxomorpholin-4-yl)-1-oxo-2-buten-1-yl]amino}-7-cyclopropylmethoxyquinazoline, 4-[(3-chloro-4-fluorophenyl)amino]-6-[2-((S)-6-methyl-2-oxomorpholin-4-yl)ethoxy]-7-methoxyquinazoline, 4-[(3-chloro-4-fluorophenyl)amino]-6-({4-[N-(2-methoxyethyl)-N-methylamino]-1-oxo-2-buten-1-yl}amino)-7-cyclopropylmethoxyquinazoline, 4-[(3-chloro-4-fluorophenyl)amino]-6-{[4-(N,N-dimethylamino)-1-oxo-2-buten-1-yl]amino}-7-cyclopentyloxyquinazoline, 4-[(3-chloro-4-fluorophenyl)amino]-6-{[4-((S)-2-methoxymethyl-6-oxomorpholin-4-yl)-1-oxo-2-buten-1-yl]amino}-7-cyclopropylmethoxyquinazoline, 4-[(R)-(1-phenylethyl)amino]-6-{[4-(N,N-bis-(2-methoxyethyl)amino)-1-oxo-2-buten-1-yl]amino}-7-cyclopropylmethoxyquinazoline, 4-[(R)-(1-phenylethyl)amino]-6-({4-[N-(2-methoxyethyl)-N-ethylamino]-1-oxo-2-buten-1-yl}amino)-7-cyclopropylmethoxyquinazoline, 4-[(R)-(1-phenylethyl)amino]-6-({4-[N-(2-methoxyethyl)-N-methylamino]-1-oxo-2-buten-1-yl}amino)-7-cyclopropylmethoxyquinazoline, 4-[(R)-(1-phenylethyl)amino]-6-({4-[N-(tetrahydropyran-4-yl)-N-methylamino]-1-oxo-2-buten-1-yl}amino)-7-cyclopropylmethoxyquinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)quinazoline, 4-[(3-chloro-4-fluorophenyl)amino]-6-({4-[N-(2-methoxyethyl)-N-methylamino]-1-oxo-2-buten-1-yl}amino)-7-cyclopentyloxyquinazoline, 4-[(3-chloro-4-fluorophenyl)amino]-6-{[4-(N-cyclopropyl-N-methylamino)-1-oxo-2-buten-1-yl]amino}-7-cyclopentyloxyquinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]quinazoline, 4-[(3-chloro-4-fluorophenyl)amino]-6-[(4-dimethylaminocyclohexyl)amino]pyrimido[5,4-d]pyrimidine, and 4-[(3-chloro-4-fluorophenyl)amino]-6-[3-(morpholin-4-yl)propyloxy]-7-methoxyquinazoline.

Particularly preferred are the EGFR kinase inhibitors 2 selected from the group consisting of 4-[(3-chloro-4-fluorophenyl)amino]-7-[4-((R)-6-methyl-2-oxomorpholin-4-yl)butyloxy]-6-[(vinylcarbonyl)amino]quinazoline, 4-[(3-chloro-4-fluorophenyl)amino]-7-[4-((S)-6-methyl-2-oxomorpholin-4-yl)butyloxy]-6-[(vinylcarbonyl)amino]quinazoline, 4-[(3-chloro-4-fluorophenyl)amino]-7-(2-{4-[(S)-(2-oxotetrahydrofuran-5-yl)-carbonyl]piperazin-1-yl}ethoxy)-6-[(vinylcarbonyl)amino]quinazoline, 4-[(3-chloro-4-fluorophenyl)amino]-7-[2-((S)-6-methyl-2-oxomorpholin-4-yl)ethoxy]-6-[(vinylcarbonyl)amino]quinazoline, 4-[(3-chloro-4-fluorophenyl)amino]-6-[(4-{N-[2-(ethoxycarbonyl)ethyl]-N-[(ethoxycarbonyl)methyl]amino}-1-oxo-2-buten-1-yl)amino]-7-cyclopropylmethoxyquinazoline, 4-[(R)-(1-phenylethyl)amino]-6-{[4-(morpholin-4-yl)-1-oxo-2-buten-1-yl]amino}-7-cyclopropylmethoxyquinazoline, and 4-[(3-chloro-4-fluorophenyl)amino]-6-[3-(morpholin-4-yl)propyloxy]-7-methoxyquinazoline.

Any reference to the abovementioned EGFR kinase inhibitors 2 also includes within the scope of the present invention a reference to any pharmacologically acceptable acid addition salts thereof which may exist.

By physiologically or pharmacologically acceptable acid addition salts which may be formed from 2 are meant according to the invention pharmaceutically acceptable salts which are selected from the salts of hydrochloric acid, hydrobromic acid, sulfuric acid, phosphoric acid, methanesulfonic acid, acetic acid, fumaric acid, succinic acid, lactic acid, citric acid, tartaric acid, or maleic acid. According to the invention, the salts of the compounds 2 selected from among the salts of acetic acid, hydrochloric acid, hydrobromic acid, sulfuric acid, phosphoric acid, and methanesulfonic acid are preferred.

The pharmaceutical combinations of 1 and 2 according to the invention are preferably administered by inhalation. Suitable inhalable powders packed into suitable capsules (inhalettes) may be administered using suitable powder inhalers. Alternatively, the drug may be inhaled by the application of suitable inhalation aerosols. These also include powdered inhalation aerosols which contain HFA134a, HFA227, or a mixture thereof as propellant gas, for example. The drug may also be inhaled using suitable solutions of the pharmaceutical combination consisting of 1 and 2.

In one aspect, therefore, the invention relates to a pharmaceutical composition which contains a combination of 1 and 2.

In another aspect, the present invention relates to a pharmaceutical composition which contains one or more salts 1 and one or more compounds 2, optionally in the form of their solvates or hydrates. The active substances may be combined in a single preparation or contained in two separate formulations. Pharmaceutical compositions which contain the active substances 1 and 2 in a single preparation are preferred according to the invention.

In another aspect, the present invention relates to a pharmaceutical composition which contains, in addition to therapeutically effective quantities of 1 and 2, a pharmaceutically acceptable carrier or excipient. In another particularly preferred aspect the present invention relates to a pharmaceutical composition which does not contain any pharmaceutically acceptable excipient in addition to therapeutically effective quantities of 1 and 2.

The present invention also relates to the use of 1 and 2 for preparing a pharmaceutical composition containing therapeutically effective quantities of 1 and 2 for treating inflammatory or obstructive diseases of the respiratory tract, particularly asthma or chronic obstructive pulmonary disease (COPD), as well as complications thereof such as pulmonary hypertension, as well as allergic and non-allergic rhinitis, provided that treatment with EGFR kinase inhibitors is not contraindicated from a therapeutic point of view, by simultaneous or successive administration.

The present invention also relates to the simultaneous or successive use of therapeutically effective doses of the combination of the above pharmaceutical compositions 1 and 2 for treating inflammatory and/or obstructive diseases of the respiratory tract, particularly asthma or chronic obstructive pulmonary disease (COPD), as well as complications thereof such as pulmonary hypertension, as well as allergic and non-allergic rhinitis, provided that treatment with EGFR kinase inhibitors is not contraindicated from a therapeutic point of view, by simultaneous or successive administration.

In the active substance combinations of 1 and 2 according to the invention, ingredients 1 and 2 may be present in the form of their enantiomers, mixtures of enantiomers or in the form of racemates.

The proportions in which the two active substances 1 and 2 may be used in the active substance combinations according to the invention are variable. Active substances 1 and 2 may possibly be present in the form of their solvates or hydrates. Depending on the choice of the compounds 1 and 2, the weight ratios which may be used within the scope of the present invention vary on the basis of the different molecular weights of the various compounds and their different potencies. As a rule, the pharmaceutical combinations according to the invention may contain compounds 1 and 2 in ratios by weight ranging from 1:300 to 60:1, preferably from 1:200 to 30:1.

In the particularly preferred pharmaceutical combinations which contain in addition to a compound of formula 1 a compound selected from among 4-[(3-chloro-4-fluorophenyl)amino]-7-[4-((R)-6-methyl-2-oxomorpholin-4-yl)butyloxy]-6-[(vinylcarbonyl)amino]quinazoline, 4-[(3-chloro-4-fluorophenyl)amino]-7-[4-((S)-6-methyl-2-oxomorpholin-4-yl)butyloxy]-6-[(vinylcarbonyl)amino]quinazoline, 4-[(3-chloro-4-fluorophenyl)amino]-7-(2-{4-[(S)-(2-oxotetrahydrofuran-5-yl)carbonyl]piperazin-1-yl}ethoxy)-6-[(vinylcarbonyl)amino]quinazoline, 4-[(3-chloro-4-fluorophenyl)amino]-7-[2-((S)-6-methyl-2-oxomorpholin-4-yl)ethoxy]-6-[(vinylcarbonyl)amino]quinazoline, 4-[(3-chloro-4-fluorophenyl)amino]-6-[(4-{N-[2-(ethoxycarbonyl)ethyl]-N-[(ethoxycarbonyl)methyl]amino}-1-oxo-2-buten-1-yl)amino]-7-cyclopropylmethoxyquinazoline, 4-[(R)-(1-phenylethyl)amino]-6-{[4-(morpholin-4-yl)-1-oxo-2-buten-1-yl]amino}-7-cyclopropylmethoxyquinazoline and 4-[(3-chloro-4-fluorophenyl)amino]-6-[3-(morpholin-4-yl)propyloxy]-7-methoxyquinazoline as EGFR kinase inhibitors 2, the weight ratios of 1 to 2 are preferably in a range wherein the cation 1′ and 2 are present in proportions ranging from 1:180 to 15:1, more preferably from 1:150 to 3:1, most preferably from 1:100 to 1:30.

The pharmaceutical compositions according to the invention containing the combinations of 1 and 2 are normally used so that 1 and 2 may be present together in doses from 1000 to 100,000 μg, preferably from 1500 to 50,000 μg, more preferably from 2000 to 10,000 μg, even more preferably from 2500 to 7500 μg per single dose. For example, combinations of 1 and 2 according to the invention contain an amount of 1′ and EGFR kinase inhibitors 2 such that the total dosage per single dose is 2500 μg, 2550 μg, 2600 μg, 2650 μg, 2700 μg, 2750 μg, 2800 μg, 2850 μg, 2900 μg, 2950 μg, 3000 μg, 3050 μg, 3100 μg, 3150 μg, 3200 μg, 3250 μg, 3300 μg, 3350 μg, 3400 μg, 3450 μg, 3500 μg, 3550 μg, 3600 μg, 3650 μg, 3700 μg, 3750 μg, 3800 μg, 3850 μg, 3900 μg, 3950 μg, 4000 μg, 4050 μg, 4100 μg, 4150 μg, 4200 μg, 4250 μg, 4300 μg, 4350 μg, 4400 μg, 4450 μg, 4500 μg, 4550 μg, 4600 μg, 4650 μg, 4700 μg, 4750 μg, 4800 μg, 4850 μg, 4900 μg, 4950 μg, 5000 μg, 5050 μg, 5100 μg, 5150 μg, 5200 μg, 5250 μg, 5300 μg, 5350 μg, 5400 μg, 5450 μg, 5500 μg, 5550 μg, 5600 μg, 5650 μg, 5700 μg, 5750 μg, 5800 μg, 5850 μg, 5900 μg, 5950 μg, 6000 μg, 6050 μg, 6100 μg, 6150 μg, 6200 μg, 6250 μg, 6300 μg, 6350 μg, 6400 μg, 6450 μg, 6500 μg, 6550 μg, 6600 μg, 6650 μg, 6700 μg, 6750 μg, 6800 μg, 6850 μg, 6900 μg, 6950 μg, 7000 μg, 7050 μg, 7100 μg, 7150 μg, 7200 μg, 7250 μg, 7300 μg, 7350 μg, 7400 μg, 7450 μg, 7500 μg, or the like. These proposed dosages per single dose are not to be regarded as being restricted to the numerical values explicitly mentioned but are merely disclosed by way of example. Obviously, dosages which fluctuate around these values within a range of about ±25 μg are also covered by the values mentioned by way of example. In these dosage ranges the active substances 1′ and 2 may be present in the weight ratios described above.

For example and without restricting the scope of the invention thereto, the combinations of 1 and 2 according to the invention may contain an amount of 1′ and EGFR kinase inhibitor 2 such that 16.5 μg of 1′ and 2500 μg of 2, 16.5 μg of 1′ and 3000 μg of 2, 16.5 μg of 1′ and 3500 μg of 2, 16.5 μg of 1′ and 4000 μg of 2, 16.5 μg of 1′ and 4500 μg of 2, 16.5 μg of 1′ and 5000 μg of 2, 16.5 μg of 1′ and 5500 μg of 2, 16.5 μg of 1′ and 6000 μg of 2, 16.5 μg of 1′ and 6500 μg of 2, 16.5 μg of 1′ and 7000 μg of 2, 33,1 μg of 1′ and 2500 μg of 2, 33.1 μg of 1′ and 3000 μg of 2, 33.1 μg of 1′ and 3500 μg of 2, 33.1 μg of 1′ and 4000 μg of 2, 33.1 μg of 1′ and 4500 μg of 2, 33.1 μg of 1′ and 5000 μg of 2, 33.1 μg of 1′ and 5500 μg of 2, 33.1 μg of 1′ and 6000 μg of 2, 33.1 μg of 1′ and 6500 μg of 2, 33.1 μg of 1′ and 7000 μg of 2, 49.5 μg of 1′ and 2500 μg of 2, 49.5 μg of 1′ and 3000 μg of 2, 49.5 μg of 1′ and 3500 μg of 2, 49.5 μg of 1′ and 4000 μg of 2, 49.5 μg of 1′ and 4500 μg of 2, 49.5 μg of 1′ and 5000 μg of 2, 49.5 μg of 1′ and 5500 μg of 2, 49.5 μg of 1′ and 6000 μg of 2, 49.5 μg of 1′ and 6500 μg of 2, 49.5 μg of 1′ and 7000 μg of 2, 82.6 μg of 1′ and 2500 μg of 2, 82.6 μg of 1′ and 3000 μg of 2, 82.6 μg of 1′ and 3500 μg of 2, 82.6 μg of 1′ and 4000 μg of 2, 82.6 μg of 1′ and 4500 μg of 2, 82.6 μg of 1′ and 5000 μg of 2, 82.6 μg of 1′ and 5500 μg of 2, 82.6 μg of 1′ and 6000 μg of 2, 82.6 μg of 1′ and 6500 μg of 2, 82.6 μg of 1′ and 7000 μg of 2, 165.1 μg of 1′ and 2500 μg of 2, 165.1 μg of 1′ and 3000 μg of 2, 165.1 μg of 1′ and 3500 μg of 2, 165.1 μg of 1′ and 4000 μg of 2, 165.1 μg of 1′ and 4500 μg of 2, 165.1 μg of 1′ and 5000 μg of 2, 165.1 μg of 1′ and 5500 μg of 2, 165.1 μg of 1′ and 6000 μg of 2, 165.1 μg of 1′ and 6500 μg of 2, 165.1 μg of 1′ and 7000 μg of 2, 206.4 μg of 1′ and 2500 μg of 2, 206.4 μg of 1′ and 3000 μg of 2, 206.4 μg of 1′ and 3500 μg of 2, 206.4 μg of 1′ and 4000 μg of 2, 206.4 μg of 1′ and 4500 μg of 2, 206.4 fig of 1′ and 5000 μg of 2, 206.4 μg of 1′ and 5500 μg of 2, 206.4 μg of 1′ and 6000 μg of 2, 206.4 μg of 1′ and 6500 μg of 2, 206.4 μg of 1′ and 7000 μg of 2, 412.8 μg of 1′ and 2500 μg of 2, 412.8 μg of 1′ and 3000 μg of 2, 412.8 μg of 1′ and 3500 μg of 2, 412.8 μg of 1′ and 4000 μg of 2, 412.8 μg of 1′ and 4500 μg of 2, 412.8 μg of 1′ and 5000 μg of 2, 412.8 μg of 1′ and 5500 μg of 2, 412.8 μg of 1′ and 6000 μg of 2, 412.8 μg of 1′ and 6500 μg of 2, or 412.8 μg of 1′ and 7000 μg of 2 are administered per single dose.

If the active substance combination wherein 1 denotes the bromide is used as the preferred combination of 1 and 2 according to the invention, the quantities of active substances 1′ and 2 administered per single dose as specified by way of example correspond to the following quantities of 1 and 2 administered per single dose: 20 μg of 1 and 2500 μg of 2 20 μg of 1 and 3000 μg of 2, 20 μg of 1 and 3500 μg of 2, 20 μg of 1 and 4000 μg of 2, 20 μg of 1 and 4500 μg of 2, 20 μg of 1 and 5000 μg of 2, 20 μg of 1 and 5500 μg of 2, 20 μg of 1 and 6000 μg of 2, 20 μg of 1 and 6500 μg of 2, 20 μg of 1 and 7000 μg of 2, 40 μg of 1 and 2500 μg of 2, 40 μg of 1 and 3000 μg of 2, 40 μg of 1 and 3500 μg of 2, 40 μg of 1 and 4000 μg of 2, 40 μg of 1 and 4500 μg of 2, 40 μg of 1 and 5000 μg of 2, 40 μg of 1 and 5500 μg of 2, 40 μg of 1 and 6000 μg of 2, 40 μg of 1 and 6500 μg of 2, 40 μg of 1 and 7000 μg of 2, 60 μg of 1 and 2500 μg of 2, 60 μg of 1 and 3000 μg of 2, 60 μg of 1 and 3500 μg of 2, 60 μg of 1 and 4000 μg of 2, 60 μg of 1 and 4500 μg of 2, 60 μg of 1 and 5000 μg of 2, 60 μg of 1 and 5500 μg of 2, 60 μg of 1 and 6000 μg of 2, 60 μg of 1 and 6500 μg of 2, 60 μg of 1 and 7000 μg of 2, 100 μg of 1 and 2500 μg of 2, 100 μg of 1 and 3000 μg of 2, 100 μg of 1 and 3500 μg of 2, 100 μg of 1 and 4000 μg of 2, 100 μg of 1 and 4500 μg of 2, 100 μg of 1 and 5000 μg of 2, 100 μg of 1 and 5500 μg of 2, 100 μg of 1 and 6000 μg of 2, 100 μg of 1 and 6500 μg of 2, 100 μg of 1 and 7000 μg of 2, 200 μg of 1 and 2500 μg of 2, 200 μg of 1 and 3000 μg of 2, 200 μg of 1 and 3500 μg of 2, 200 μg of 1 and 4000 μg of 2, 200 μg of 1 and 4500 μg of 2, 200 μg of 1 and 5000 μg of 2, 200 μg of 1 and 5500 μg of 2, 200 μg of 1 and 6000 μg of 2, 200 μg of 1 and 6500 μg of 2, 200 μg of 1 and 7000 μg of 2, 250 μg of 1 and 2500 μg of 2, 250 μg of 1 and 3000 μg of 2, 250 μg of 1 and 3500 μg of 2, 250 μg of 1 and 4000 μg of 2, 250 μg of 1 and 4500 μg of 2, 250 μg of 1 and 5000 μg of 2, 250 μg of 1 and 5500 μg of 2, 250 μg of 1 and 6000 μg of 2, 250 μg of 1 and 6500 μg of 2 or 250 μg of 1 and 7000 μg of 2, 500 μg of 1 and 2500 μg of 2, 500 μg of 1 and 3000 μg of 2, 500 μg of 1 and 3500 μg of 2, 500 μg of 1 and 4000 μg of 2, 500 μg of 1 and 4500 μg of 2, 500 μg of 1 and 5000 μg of 2, 500 μg of 1 and 5500 μg of 2, 500 μg of 1 and 6000 μg of 2, 500 μg of 1 and 6500 μg of 2, or 500 μg of 1 and 7000 μg of 2.

The active substance combinations of 1 and 2 according to the invention are preferably administered by inhalation. For this purpose, ingredients 1 and 2 have to be made available in forms suitable for inhalation. Inhalable preparations include inhalable powders, propellant-containing metering aerosols or propellant-free inhalable solutions.

Inhalable powders according to the invention containing the combination of active substances 1 and 2 may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1 and 2 either together in one formulation or in two or three separate formulations. These formulations which may be used within the scope of the present invention are described in more detail in the next part of the specification.

A) Inhalable Powder Containing the Combinations of Active Substances 1 and 2 According to the Invention:

The inhalable powders according to the invention may contain 1 and 2 either on their own or in admixture with suitable physiologically acceptable excipients.

If the active substances 1 and 2 are present in admixture with physiologically acceptable excipients, the following physiologically acceptable excipients may be used to prepare these inhalable powders according to the invention: monosaccharides (e.g., glucose or arabinose), disaccharides (e.g., lactose, saccharose, maltose, trehalose), oligo- and polysaccharides (e.g., dextran), polyalcohols (e.g., sorbitol, mannitol, xylitol), salts (e.g., sodium chloride, calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipient mentioned above. These finer excipients are also selected from the group of possible excipients listed hereinbefore. Finally, in order to prepare the inhalable powders according to the invention, micronized active substance 1 and 2, preferably with an average particle size of 0.5 to 10 μm, more preferably from 1 to 6 μm, is added to the excipient mixture. Processes for producing the inhalable powders according to the invention by grinding and micronizing and by finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630, or by other means as described in DE 36 25 685 A. Preferably, the inhalable powders according to the invention which contain physiologically acceptable excipients in addition to 1 and 2 are packed into capsules (to produce so-called inhalettes) which are used in inhalers as described, for example, in WO 94/28958.

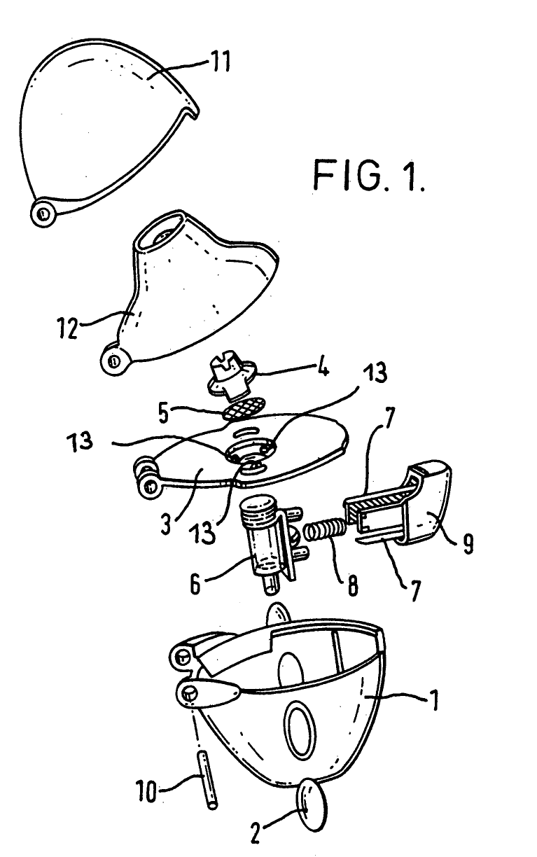

A particularly preferred inhaler for using the pharmaceutical combination according to the invention in inhalettes is shown in FIG. 1. This inhaler (HANDIHALER®) for inhaling powdered pharmaceutical compositions from capsules is characterized by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, as well as air through-holes 13 for adjusting the flow resistance.

If the inhalable powders according to the invention are to be packed into capsules (inhalettes) for the preferred use described above, the quantities packed into each capsule should be 1 to 50 mg, preferably 3 to 45 mg, more particularly 5 to 40 mg of inhalable powder per capsule. These capsules contain, according to the invention, either together or separately, the doses of 1′ and 2 mentioned hereinbefore for each single dose.

B) Propellant Gas-Driven Inhalation Aerosols Containing the Combinations of Active Substances 1 and 2 According to the Invention:

Inhalation aerosols containing propellant gas according to the invention may contain 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as chlorinated and/or fluorinated derivatives of methane, ethane, propane, butane, cyclopropane or cyclobutane.

The propellant gases mentioned above may be used on their own or in mixtures thereof.

Particularly preferred propellant gases are halogenated alkane derivatives selected from TG11, TG12, TG134a and TG227. Of the abovementioned halogenated hydrocarbons, TG134a (1,1,1,2-tetrafluoroethane) and TG227 (1,1,1,2,3,3,3-heptafluoropropane) and mixtures thereof are preferred according to the invention.

The propellant-driven inhalation aerosols according to the invention may also contain other ingredients such as co-solvents, stabilizers, surfactants, antioxidants, lubricants and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1 and/or 2.

If the active substances 1 and/or 2 are present in dispersed form, the particles of active substance preferably have an average particle size of up to 10 μm, preferably from 0.1 to 5 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterized in that they contain the propellant gas-containing aerosols described above according to the invention. The present invention also relates to cartridges which are fitted with a suitable valve and can be used in a suitable inhaler and which contain one of the abovementioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions or Suspensions Containing the Combinations of Active Substances 1 and 2 According to the Invention:

It is particularly preferred to use the active substance combination according to the invention in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulfuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulfuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g., as flavorings, antioxidants or complexing agents, such as citric acid or ascorbic acid, for example.

According to the invention, it is particularly preferred to use hydrochloric acid to adjust the pH.

According to the invention, the addition of edetic acid (EDTA) or one of the known salts thereof, sodium edetate, as stabilizer or complexing agent is unnecessary in the present formulation. Other embodiments may contain this compound or these compounds. In a preferred embodiment the content based on sodium edetate is less than 100 mg/100 mL, preferably less than 50 mg/100 mL, more preferably less than 20 mg/100 mL. Generally, inhalable solutions in which the content of sodium edetate is from 0 to 10 mg/100 mL are preferred.

Co-solvents and/or other excipients may be added to the propellant-free inhalable solutions according to the invention. Preferred co-solvents are those which contain hydroxyl groups or other polar groups, e.g., alcohols—particularly isopropyl alcohol, glycols—particularly propyleneglycol, polyethyleneglycol, polypropylene glycol, glycol ether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilizers, complexing agents, antioxidants and/or preservatives which guarantee or prolong the shelf life of the finished pharmaceutical formulation, flavorings, vitamins and/or other additives known in the art. The additives also include physiologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 mL, more preferably between 5 and 20 mg/100 mL.

Preferred formulations contain, in addition to the solvent water and the combination of active substances 1 and 2, only benzalkonium chloride and sodium edetate. In another preferred embodiment, no sodium edetate is present.

The propellant-free inhalable solutions according to the invention are administered in particular using inhalers of the kind which are capable of nebulizing a small amount of a liquid formulation in the required therapeutic dose within a few seconds to produce an aerosol suitable for therapeutic inhalation. Within the scope of the present invention, preferred nebulizer are those in which a quantity of less than 100 μL, preferably less than 50 μL, more preferably between 20 and 30 μL of active substance solution can be nebulized in preferably one spray action to form an aerosol with an average particle size of less than 20 μm, preferably less than 1 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf in particular FIGS. 6a and 6b).

The nebulizers (devices) described therein are known by the name Respimat®. This nebulizer (Respimat®) can advantageously be used to produce the inhalable aerosols according to the invention containing the combination of active substances 1 and 2. Because of its cylindrical shape and handy size of less than 9 to 15 cm long and 2 to 4 cm wide, this device can be carried at all times by the patient. The nebulizer sprays a defined volume of the pharmaceutical formulation at high pressures through small nozzles so as to produce inhalable aerosols.

The preferred atomizer essentially consists of an upper housing part, a pump housing, a nozzle, a locking mechanism, a spring housing, a spring and a storage container, characterized by
a pump housing which is secured in the upper housing part and which comprises at one end a nozzle body with the nozzle or nozzle arrangement,
a hollow plunger with valve body,
a power takeoff flange in which the hollow plunger is secured and which is located in the upper housing part,
a locking mechanism situated in the upper housing part,
a spring housing with the spring contained therein, which is rotatably mounted on the upper housing part by means of a rotary bearing,
a lower housing part which is fitted onto the spring housing in the axial direction.

The hollow plunger with valve body corresponds to a device disclosed in WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 to 60 MPa (about 50 to 600 bar), preferably 10 to 60 MPa (about 100 to 600 bar) on the fluid, the measured amount of active substance solution, at its high pressure end at the moment when the spring is actuated. Volumes of 10 to 50 microliters are preferred, while volumes of 10 to 20 microliters are particularly preferred and a volume of 15 microliters per spray is most particularly preferred. The valve body is preferably mounted at the end of the hollow plunger facing the valve body.

The nozzle in the nozzle body is preferably microstructured, i.e., produced by microtechnology. Microstructured valve bodies are disclosed for example in WO-94/07607; reference is hereby made to the contents of this specification, particularly FIG. 1 therein and the associated description.

The nozzle body consists, for example, of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20 to 160° to one another, preferably 60 to 150°, most preferably 80 to 100°. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 to 300 bar, and is atomized into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g., a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomizer axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomizer; this causes the deformable ring to deform in the annular plane. Details of the construction of the locking mechanism are given in WO 97/20590.

The lower housing part is pushed axially over the spring housing and covers the mounting, the drive of the spindle and the storage container for the fluid.

When the atomizer is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360 degrees, e.g., 180 degrees. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomized may be pushed into the atomizer one after another and used in succession. The storage container contains the aqueous aerosol preparation according to the invention. The atomizing process is initiated by pressing gently on the actuating button. As a result, the locking mechanism opens up the path for the power takeoff member. The biased spring pushes the plunger into the cylinder of the pump housing. The fluid leaves the nozzle of the atomizer in atomized form.

Further details of construction are disclosed in PCT Applications WO 97/12683 and WO 97/20590, to which reference is hereby made.

The components of the atomizer (nebulizer) are made of a material which is suitable for its purpose. The housing of the atomizer and, if its operation permits, other parts as well, are preferably made of plastics, e.g., by injection molding. For medicinal purposes, physiologically safe materials are used.

FIGS. 6a/b of WO 97/12687, to which reference is explicitly made at this point, show the nebulizer (Respimat®) which can advantageously be used for inhaling the aqueous aerosol preparations according to the invention. FIG. 6a of WO 97/12687 shows a longitudinal section through the atomizer with the spring biased while FIG. 6b of WO 97/12687 shows a longitudinal section through the atomizer with the spring relaxed. The upper housing part (51) contains the pump housing (52) on the end of which is mounted the holder (53) for the atomizer nozzle. In the holder is the nozzle body (54) and a filter (55). The hollow plunger (57) fixed in the power takeoff flange (56) of the locking mechanism projects partially into the cylinder of the pump housing. At its end the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomized. The storage container is sealed off by the stopper (73) through which the hollow plunger projects into the storage container and is immersed at its end in the fluid (supply of active substance solution). The spindle (74) for the mechanical counter is mounted in the covering of the spring housing. At the end of the spindle facing the upper housing part is the drive pinion (75). The slider (76) sits on the spindle.

The nebulizer described above is suitable for nebulizing the aerosol preparations according to the invention to produce an aerosol suitable for inhalation.

If the formulation according to the invention is nebulized using the technology described above (Respimat®) the quantity delivered should correspond to a defined quantity with a tolerance of not more than 25%, preferably 20% of this amount in at least 97%, preferably at least 98% of all operations of the inhaler (spray actuations). Preferably, between 5 and 30 mg of formulation, most preferably between 5 and 20 mg of formulation are delivered as a defined mass on each actuation.

However, the formulation according to the invention may also be nebulized by means of inhalers other than those described above, e.g., jet stream inhalers.

Accordingly, in a further aspect, the invention relates to pharmaceutical formulations in the form of propellant-free inhalable solutions or suspensions as described above combined with a device suitable for administering these formulations, preferably in conjunction with the Respimat®. Preferably, the invention relates to propellant-free inhalable solutions or suspensions characterized by the combination of active substances 1 and 2 according to the invention in conjunction with the device known by the name Respimat®. In addition, the present invention relates to the abovementioned devices for inhalation, preferably the Respimat®, characterized in that they contain the propellant-free inhalable solutions or suspensions according to the invention as described hereinbefore.

The propellant-free inhalable solutions or suspensions according to the invention may take the form of concentrates or sterile inhalable solutions or suspensions ready for use, as well as the abovementioned solutions and suspensions designed for use in a Respimat®. Formulations ready for use may be produced from the concentrates, for example, by the addition of isotonic saline solutions. Sterile formulations ready for use may be administered using energy-operated fixed or portable nebulizers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterized in that the device is an energy-operated free-standing or portable nebulizer which produces inhalable aerosols by means of ultrasound or compressed air by the Venturi principle or other methods.

The Examples which follow serve to illustrate the present invention in more detail without restricting the scope of the invention to the following embodiments by way of example.

Starting Materials

In order to prepare compounds 2 mentioned within the scope of the present invention and not yet known in the art:

### I.) 4-[(3-chloro-4-fluorophenyl)amino]-7-{3-[4-(2-oxotetrahydrofuran-4-yl)piperazin-1-yl]propyloxy}-6-[(vinylcarbonyl)amino]quinazoline

A mixture of 166 mg of acrylic acid and 0.77 mL of triethylamine in 10 mL of tetrahydrofuran is cooled to −50° C. in a dry ice/acetone cooling bath and combined with a solution of 175 μL of acrylic acid chloride in 4 mL of tetrahydrofuran. The reaction mixture is stirred at this temperature for 45 minutes. Then a solution of 427 mg of 6-amino-4-[(3-chloro-4-fluorophenyl)amino]-7-{3-[4-(2-oxotetrahydrofuran-4-yl)piperazin-1-yl]propyloxy}quinazoline in 10 mL of tetrahydrofuran is added dropwise within 20 minutes. The reaction mixture is then slowly allowed to warm up to 0° C. and stirred at this temperature until the reaction is complete. It is then combined with ice water whereupon a viscous precipitate is formed. This is extracted thoroughly several times with ethyl acetate/methanol. The combined organic phases are washed with saturated sodium chloride solution, dried over magnesium sulfate and evaporated down. The yellowish, resinous crude product is purified by chromatography over a silica gel column with methylene chloride/methanol (95:5) as eluant. Yield: 148 mg (31% of theory); R_{f} value: 0.45 (silica gel, methylene chloride/methanol/concentrated aqueous ammonia solution=90:10:0.1); mass spectrum (ESI⁺): m/z=567, 569 [M−H]⁺.

The following compound is obtained analogously to I.):

### 4-[(3-chloro-4-fluorophenyl)amino]-7-(2-{4-[(S)-(2-oxotetrahydrofuran-5-yl)carbonyl]-piperazin-1-yl}ethoxy)-6-[(vinylcarbonyl)amino]quinazoline

R_{f} value: 0.46 (silica gel, methylene chloride/methanol/concentrated aqueous ammonia solution=90:10:0.1); mass spectrum (ESI⁺): m/z=581, 583 [M−H]⁺.

### II.) 4-[(3-chloro-4-fluorophenyl)amino]-7-[3-(2,2-dimethyl-6-oxomorpholin-4-yl)propyloxy]-6-[(vinylcarbonyl)amino]quinazoline

0.47 mL triethylamine is added to 101 mg of acrylic acid in 5 mL of tetrahydrofuran under a nitrogen atmosphere. This mixture is cooled to about −50° C. in a dry ice/acetone cooling bath and combined with 119 mg of acrylic acid chloride in 3 mL of tetrahydrofuran, whereupon a colorless precipitate is formed. The suspension is stirred for about another hour at this temperature. Then 240 mg of 6-amino-4-[(3-chloro-4-fluorophenyl)amino]-7-[3-(2,2-dimethyl-6-oxomorpholin-4-yl)propyloxy]quinazoline in 7 mL of tetrahydrofuran are added dropwise at −55° C. The reaction mixture is allowed to heat up slowly to −30° C. After about an hour, the dry ice/acetone cooling bath is exchanged for an ice/sodium chloride cooling bath. The reaction mixture is then allowed to come up to 0° C. therein. As soon as the reaction is complete, the reaction mixture is combined with water and methylene chloride and made alkaline with sodium hydroxide solution. The aqueous phase separated off is extracted again with methylene chloride and a little methanol. The combined organic extracts are washed with water, dried, and evaporated down. A yellow resin remains which is chromatographed through a silica gel column with methylene chloride/methanol (98:2) as eluant. The desired product is stirred with a little tert-butylmethyl ether, the fine crystalline precipitate is suction filtered, washed again with tert-butylmethyl ether and dried in vacuo at 50° C. Yield: 160 mg (60% of theory); R_{f} value: 0.42 (silica gel, methylene chloride/methanol=95:5); mass spectrum (ESI⁺): m/z=526, 528 [M−H]⁺.

The following compounds are obtained analogously to II.):

### (1) 4-[(3-chloro-4-fluorophenyl)amino]-7-[2-((S)-6-methyl-2-oxomorpholin-4-yl)ethoxy]-6-[(vinylcarbonyl)amino]quinazoline

R_{f} value: 0.32 (silica gel, methylene chloride/methanol=95:5); mass spectrum (ESI⁺): m/z=498, 500 [M−H]⁺.

### (2) 4-[(3-chloro-4-fluorophenyl)amino]-7-[4-((R)-6-methyl-2-oxomorpholin-4-yl)butyl-oxy]-6-[(vinylcarbonyl)amino]quinazoline

R_{f} value: 0.30 (silica gel, methylene chloride/methanol=95:5); mass spectrum (ESI⁺): m/z=550, 552 [M+Na]⁺.

### (3) 4-[(3-chloro-4-fluorophenyl)amino]-7-[4-((S)-6-methyl-2-oxomorpholin-4-yl)butyl-oxy]-6-[(vinylcarbonyl)amino]quinazoline

Mass spectrum (ESI⁺): m/z=526, 528 [M−H]⁺.

### III.) 4-[(3-chloro-4-fluorophenyl)amino]-6-{[4-(N,N-diethylamino)-1-oxo-2-buten-1-yl]amino}-7-cyclopropylmethoxyquinazoline

0.67 mL of oxalyl chloride and one drop of dimethylformamide are added at ambient temperature to a solution of 640 mg of 4-bromo-2-butenoic acid in 10 mL of methylene chloride. The reaction mixture is stirred for about another half hour at ambient temperature until the development of gas has ended. The acid chloride produced is largely freed from solvent using the rotary evaporator in vacuo. Then the crude product is dissolved in 10 mL of methylene chloride and added dropwise while cooling with an ice bath to a mixture of 1.00 g of 6-amino-4-[(3-chloro-4-fluorophenyl)amino]7-cyclopropylmethoxyquinazoline and 1.60 mL of Hünig base in 50 mL of tetrahydrofuran. The reaction mixture is stirred for 1.5 hours in the ice bath and for a further 2 hours at ambient temperature. Then 2.90 mL of diethylamine is added and the mixture is stirred for 2.5 days at ambient temperature. For working up, the reaction mixture is filtered and the filtrate is evaporated down. The flask residue is purified by chromatography over a silica gel column with ethyl acetate/methanol (19:1). Yield: 550 mg (40% of theory); melting point: 114° C.; mass spectrum (ESI⁺): m/z=498, 500 [M+H]⁺.

The following compounds are obtained analogously to III.):

### (1) 4-[(3-chloro-4-fluorophenyl)amino]-6-{[4-(morpholin-4-yl)-1-oxo-2-buten-1-yl]-amino}-7-cyclopropylmethoxyquinazoline

R_{f} value: 0.53 (silica gel, ethyl acetate/methanol=9:1); mass spectrum (ESI⁺): m/z=510, 512 [M−H]⁺.

### (2) 4-[(3-chloro-4-fluorophenyl)amino]-6-{[4-(N,N-dimethylamino)-1-oxo-2-buten-1-yl]-amino}-7-cyclopropylmethoxyquinazoline

Melting point: 137° C.; mass spectrum (ESI⁺): m/z=470, 472 [M+H]⁺.

### (3) 4-[(R)-(1-phenylethyl)amino]-6-{[4-(morpholin-4-yl)-1-oxo-2-buten-1-yl]amino}-7-cyclopropylmethoxyquinazoline

R_{f} value: 0.37 (silica gel, ethyl acetate/methanol=9:1); mass spectrum (ESI⁺): m/z=488 [M+H]⁺.

### (4) 4-[(R)-(1-phenylethyl)amino]-6-{[4-(morpholin-4-yl)-1-oxo-2-buten-1-yl]amino}-7-cyclopentyloxyquinazoline

R_{f} value: 0.35 (silica gel, ethyl acetate/methanol=9:1); mass spectrum (ESI⁺): m/z=502 [M+H]⁺.

### IV.) 4-[(3-methylphenyl)amino]-6-[(4-{N-[(ethoxycarbonyl)methyl]-N-methylamino}-1-oxo-2-buten-1-yl)amino]-7-methoxyquinazoline

0.86 mL of oxalyl chloride and one drop of dimethylformamide are added to a solution of 842 mg of 4-bromo-2-butenoic acid in 15 mL of methylene chloride at ambient temperature. The reaction mixture is stirred for about another hour at ambient temperature until the development of gas has ended. The acid chloride formed is largely freed from solvent in vacuo using the rotary evaporator. Then the crude product is taken up in 10 mL of methylene chloride and added dropwise within five minutes to a mixture of 1.0 g of 6-amino-4-[(3-methylphenyl)amino]-7-methoxyquinazoline and 2.0 mL of Hünig base in 50 mL of tetrahydrofuran while cooling with an ice bath. The reaction mixture is stirred for two hours while cooling with an ice bath and then for another two hours at ambient temperature. Then 6.7 mL of Hünig base, 5.48 g of sarcosine ethylester hydrochloride, and 3 mL of dimethylformamide are added and the whole is stirred overnight at ambient temperature. For working up the reaction mixture is evaporated down in vacuo using the rotary evaporator and the flask residue is distributed between 75 mL of ethyl acetate and 75 mL of water. The organic phase is washed with water and saturated sodium chloride solution, dried over magnesium sulfate and evaporated down. The crude product is purified by chromatography over a silica gel column with methylene chloride/methanol (20:1). Yield: 326 mg (20% of theory); melting point: 122° C.-124° C.; mass spectrum (ESI⁺): m/z=464 [M+H]⁺.

The following compound is obtained analogously to IV.):

### 4-[(3-chloro-4-fluorophenyl)amino]-6-[(4-{N-[2-(ethoxycarbonyl)ethyl]-N-[(ethoxycarbonyl)methyl]amino}-1-oxo-2-buten-1-yl)amino]-7-cyclopropylmethoxyquinazoline

R_{f} value: 0.62 (aluminum oxide, cyclohexane/ethyl acetate=1:1); mass spectrum (EI): m/z=627, 629 [M]⁺.

### V.) 4-[(3-chloro-4-fluorophenyl)amino]-6-{[4-((R)-2-methoxymethyl-6-oxomorpholin-4-yl)-1-oxo-2-buten-1-yl]amino}-7-cyclopropylmethoxyquinazoline

950 mg of 4-[(3-chloro-4-fluorophenyl)amino]-6-[(4-{N-[(ethoxycarbonyl)methyl]-N-((R)-2-hydroxy-3-methoxypropyl)amino}-1-oxo-2-buten-1-yl)amino]-7-cyclopropylmethoxyquinazoline and 195 μL of methanesulfonic acid in 10 mL of acetonitrile is refluxed for about four hours. For working up, the reaction mixture is cooled in a bath of ice water, combined with 75 mL of ethyl acetate and 25 mL of saturated sodium hydrogen carbonate solution and stirred vigorously for 10 minutes. The organic phase is separated off, washed with saturated sodium hydrogen carbonate solution, and saturated sodium chloride solution and dried over magnesium sulfate. The solvent is distilled off in vacuo, leaving a brownish foam. Yield: 610 mg (69% of theory); R_{f} value: 0.55 (silica gel, methylene chloride/methanol=9:1); mass spectrum (ESI⁺): m/z=570, 572 [M+H]⁺.

### VI.) 4-[(3-chloro-4-fluorophenyl)amino]-6-{[4-((5)-6-methyl-2-oxomorpholin-4-yl-1-oxo-2-buten-1-yl]amino}-7-cyclopropylmethoxyquinazoline

A mixture of 700 mg of 4-[(3-chloro-4-fluorophenyl)amino]-6-[(4-{N-[(tert-butyl-oxycarbonyl)methyl]-N-((S)-2-hydroxyprop-1-yl)amino}-1-oxo-2-buten-1-yl)amino]-7-cyclopropylmethoxyquinazoline and 228 mg of p-toluenesulfonic acid hydrate in 20 mL of acetonitrile is refluxed for five hours. Then a further 200 mg of p-toluenesulfonic acid hydrate is added and the mixture is again refluxed for five hours. For working up, the reaction mixture is evaporated to dryness. The flask residue is distributed between ethyl acetate and saturated sodium carbonate solution. The organic phase is separated off, washed with saturated sodium carbonate solution, water and saturated sodium chloride solution, dried over magnesium sulfate and evaporated down. The oily residue is brought to crystallization by stirring with 15 mL of diethyl ether. Melting point: 173° C.-175° C.; mass spectrum (ESI⁺): m/z=540, 542 [M+H]⁺.

The following compounds are obtained analogously to VI.):

### (1) 4-[(3-chloro-4-fluorophenyl)amino]-6-{[4-((R)-6-methyl-2-oxomorpholin-4-yl)-1-oxo-2-buten-1-yl]amino}-7-cyclopropylmethoxyquinazoline

R_{f} value: 0.54 (silica gel, methylene chloride/methanol=9:1); mass spectrum (ESI⁺): m/z=540, 542 [M+H]⁺.

### (2) 4-[(3-chloro-4-fluorophenyl)amino]-6-{[4-((R)-6-methyl-2-oxomorpholin-4-yl)-1-oxo-2-buten-1-yl]amino}-7-[(S)-(tetrahydrofuran-3-yl)oxy]quinazoline

(The reaction is carried out with methanesulfonic acid in acetonitrile). R_{f} value: 0.38 (silica gel, methylene chloride/methanol=9:1); mass spectrum (ESI⁺): m/z=556, 558 [M+H]⁺.

### VII.) 4-[(3-bromophenyl)amino]-6-[2-((S)-6-methyl-2-oxomorpholin-4-yl)ethoxy]-7-methoxyquinazoline

90 μL of methanesulfonic acid is added to 380 mg of 4-[(3-bromophenyl)amino]-6-(2-{N-[(tert-butyloxycarbonyl)methyl]-N-((S)-2-hydroxypropyl)amino}ethoxy)-7-methoxyquinazoline in 8 mL of acetonitrile. The reaction mixture is refluxed for about three hours, then another equivalent of methanesulfonic acid is added and refluxing is continued until the reaction is complete. For working up, the reaction mixture is diluted with ethyl acetate and washed with saturated sodium hydrogen carbonate solution and saturated sodium chloride solution. The organic phase is dried over magnesium sulfate and evaporated down in vacuo. The flask residue is stirred with diethyl ether and suction filtered. The title compound is obtained as a white solid. Yield: 280 mg (85% of theory); melting point: 190° C.; mass spectrum (ESI⁺): m/z=485, 487 [M−H]⁺.

The following compound is obtained analogously to VII.):

### 4-[(3-chloro-4-fluorophenyl)amino]-6-[2-((S)-6-methyl-2-oxomorpholin-4-yl)ethoxy]-7-methoxyquinazoline

(The reaction is carried out with trifluoroacetic acid in acetonitrile) melting point: 212° C.-213° C.; mass spectrum (ESI⁺): m/z=461, 463 [M+H]⁺.

### VIII.) 4-[(3-chloro-4-fluorophenyl)amino]-6-([4-[N-(2-methoxyethyl)-N-methylamino]-1-oxo-2-buten-1-yl}amino)7-cyclopropylmethoxyquinazoline

4.70 mL of oxalyl chloride is added dropwise to a solution of 4.50 g of bromocrotonic acid in 60 mL of methylene chloride. Then one drop of N,N-dimethylformamide is added. After about 30 minutes, the development of gas has ended and the reaction mixture is evaporated down in the rotary evaporator. The crude bromocrotonic acid chloride is taken up in 30 mL of methylene chloride and added dropwise to a solution of 7.00 g of 4-[(3-chloro-4-fluorophenyl)amino]-6-amino-7-cyclopropylmethoxyquinazoline and 10.20 mL of Hünig base in 150 mL of tetrahydrofuran while cooling with an ice bath. The reaction mixture is stirred for about 1.5 hours while cooling with an ice bath and for a further two hours at ambient temperature. Then 5.20 g of N-(2-methoxyethyl)-N-methylamine is added and the reaction mixture is stirred overnight at ambient temperature. For working up it is diluted with methylene chloride and washed thoroughly with water. The organic phase is dried over magnesium sulfate and evaporated down. The crude product is purified by chromatography over a silica gel column with ethyl acetate followed by ethyl acetate/methanol (19:1) as eluant. Yield: 5.07 g (51% of theory); mass spectrum (ESI⁺): m/z=512, 514 [M−H]⁺; R_{f} value: 0.25 (silica gel, ethyl acetate/methanol=9:1).

The following compounds are obtained analogously to VIII):

### (1) 4-[(3-chloro-4-fluorophenyl)amino]-6-{[4-(N,N-dimethylamino)-1-oxo-2-buten-1-yl]-amino}-7-cyclopentyloxyquinazoline

Mass spectrum (ESI⁺): m/z=482, 484 [M−H]⁺; R_{f} value: 0.11 (silica gel, ethyl acetate/methanol=9:1).

### (2) 4-[(R)-(1-phenylethyl)amino]-6-{[4-(N,N-bis-(2-methoxyethyl)amino)-1-oxo-2-buten-1-yl]amino}-7-cyclopropylmethoxyquinazoline

Mass spectrum (ESI⁺): m/z=532 [M−H]⁺; R_{f} value: 0.40 (silica gel, ethyl acetate/methanol=9:1).

### (3) 4-[(R)-(1-phenylethyl)amino]-6-({4-[N-(2-methoxyethyl)-N-ethylamino]-1-oxo-2-buten-1-yl}amino)-7-cyclopropylmethoxyquinazoline

Mass spectrum (ESI⁺): m/z=502 [M−H]⁺; R_{f} value: 0.20 (silica gel, ethyl acetate/methanol=9:1).

### (4) 4-[(R)-(1-phenylethyl)amino]-6-({4-[N-(2-methoxyethyl)-N-methylamino]-1-oxo-2-buten-1-yl}amino)-7-cyclopropylmethoxyquinazoline

Mass spectrum (ESI⁺): m/z=488 [M−H]⁺; R_{f} value: 0.25 (silica gel, ethyl acetate/methanol=9:1).

### (5) 4-[(R)-(1-phenylethyl)amino]-6-({4-[N-(tetrahydropyran-4-yl)-N-methylamino]-1-oxo-2-buten-1-yl}amino)-7-cyclopropylmethoxyquinazoline

Mass spectrum (ESI⁺): m/z=514 [M−H]⁺; R_{f} value: 0.15 (silica gel, ethyl acetate/methanol=9:1).

### (6) 4-[(3-chloro-4-fluorophenyl)amino]-6-{[4-(N,N-dimethylamino)-1-oxo-2-buten-1-yl]-amino}-7-((R)-tetrahydrofuran-3-yloxy)quinazoline

Mass spectrum (ESI⁺): m/z=486, 488 [M+H]⁺.

### (7) 4-[(3-chloro-4-fluorophenyl)amino]-6-{[4-(N,N-dimethylamino)-1-oxo-2-buten-1-yl]-amino}-7-((S)-tetrahydrofuran-3-yloxy)quinazoline

Mass spectrum (ESI⁺): m/z=486, 488 [M+H]⁺; R_{f} value: 0.45 (silica gel, methylene chloride/methanol=5:1).

### (8) 4-[(3-chloro-4-fluorophenyl)amino]-6-({4-[N-(2-methoxyethyl)-N-methylamino]-1-oxo-2-buten-1-yl}amino)-7-cyclopentyloxyquinazoline

Mass spectrum (ESI⁺): m/z=528, 530 [M−H]⁺; R_{f} value: 0.25 (silica gel, ethyl acetate/methanol=9:1).

### (9) 4-[(3-chloro-4-fluorophenyl)amino]-6-{[4-(N-cyclopropyl-N-methylamino)-1-oxo-2-buten-1-yl]amino}-7-cyclopentyloxyquinazoline

Mass spectrum (ESI⁺): m/z=508, 510 [M−H]⁺; melting point: 140° C.

### (10) 4-[(3-chloro-4-fluorophenyl)amino]-6-{[4-(N,N-dimethylamino)-1-oxo-2-buten-1-yl]-amino}-7-[(R)-(tetrahydrofuran-2-yl)methoxy]quinazoline

Mass spectrum (ESI⁺): m/z=500, 502 [M+H]⁺; melting point: 110° C.-112° C.

### (11) 4-[(3-chloro-4-fluorophenyl)amino]-6-{[4-(N,N-dimethylamino)-1-oxo-2-buten-1-yl]-amino}-7-[(S)-(tetrahydrofuran-2-yl)methoxy]quinazoline

Mass spectrum (ESI⁺): m/z=500, 502 [M+H]⁺; R_{f} value: 0.23 (silica gel, ethyl acetate/methanol/conc. aqueous ammonia=90:10:0.1).

Some particularly preferred formulations according to the invention containing the two components 1 and 2 are described hereinafter without restricting the core of the invention thereto.

### FORMULATION EXAMPLES

Inhalable Powders:

| Ingredients | μg per capsule |
|---|---|
| 1) | |
| 1 ′- bromide | 60 |
| EGFR kinase inhibitor 2 | 3500 |
| Lactose | 3440 |
| Total | 7000 |
| 2) | |
| 1 ′- bromide | 100 |
| EGFR kinase inhibitor 2 | 3000 |
| Lactose | 3900 |
| Total | 7000 |
| 3) | |
| 1 ′- bromide | 150 |
| EGFR kinase inhibitor 2 | 5000 |
| Lactose | 4850 |
| Total | 10000 |

1. A pharmaceutical composition comprising:
(a) an anticholinergic of formula 1 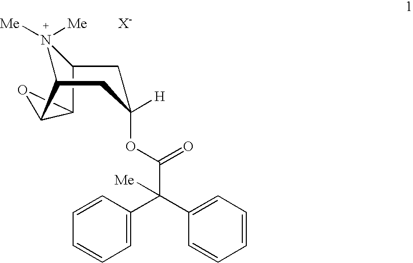
wherein X⁻ is an anion with a single negative charge; and
(b) an EGFR kinase inhibitor, wherein the EGFR kinase inhibitor is optionally in the form of an enantiomer, a mixture of enantiomers, a racemate, a solvate, or a hydrate thereof,
optionally together with one or more pharmaceutically acceptable excipients.

2. The pharmaceutical composition according to claim 1, wherein X⁻ is an anion selected from chloride, bromide, iodide, sulfate, phosphate, methanesulfonate, nitrate, maleate, acetate, citrate, fumarate, tartrate, oxalate, succinate, benzoate, and p-toluenesulfonate.

3. The pharmaceutical composition according to claim 1, wherein X⁻ is a negatively charged anion selected from chloride, bromide, 4-toluenesulfonate, and methanesulfonate.

4. The pharmaceutical composition according to claim 1, wherein X⁻ is bromide.

5. The pharmaceutical composition according to claim 1, wherein the EGFR kinase inhibitor is selected from:
4-[(3-chloro-4-fluorophenyl)amino]-7-(2-{4-[(S)-(2-oxotetrahydrofuran-5-yl)-carbonyl]piperazin-1-yl}ethoxy)-6-[(vinylcarbonyl)amino]quinazoline;
4-[(3-chloro-4-fluorophenyl)amino]-7-[2-((S)-6-methyl-2-oxomorpholin-4-yl)ethoxy]-6-[(vinylcarbonyl)amino]quinazoline;
4-[(3-chloro-4-fluorophenyl)amino]-7-[4-((R)-6-methyl-2-oxomorpholin-4-yl)butyloxy]-6-[(vinylcarbonyl)amino]quinazoline;
4-[(3-chloro-4-fluorophenyl)amino]-7-[4-((S)-6-methyl-2-oxomorpholin-4-yl)butyloxy]-6-[(vinylcarbonyl)amino]quinazoline;
4-[(3-chloro-4-fluorophenyl)amino]-6-{[4-(morpholin-4-yl)-1-oxo-2-buten-1-yl]amino}-7-cyclopropylmethoxyquinazoline;
4-[(3-chloro-4-fluorophenyl)amino]-6-{[4-(N,N-diethylamino)-1-oxo-2-buten-1-yl]-amino}-7-cyclopropylmethoxyquinazoline;
4-[(3-chloro-4-fluorophenyl)amino]-6-{[4-(N,N-dimethylamino)-1-oxo-2-buten-1-yl]amino}-7-cyclopropylmethoxyquinazoline;
4-[(3-chloro-4-fluorophenyl)amino]-6-[(4-{N-[2-(ethoxycarbonyl)ethyl]-N-[(ethoxycarbonyl)methyl]amino}-1-oxo-2-buten-1-yl)amino]-7-cyclopropylmethoxyquinazoline;
4-[(R)-(1-phenylethyl)amino]-6-{[4-(morpholin-4-yl)-1-oxo-2-buten-1-yl]amino}-7-cyclopropylmethoxyquinazoline;
4-[(R)-(1-phenylethyl)amino]-6-{[4-(morpholin-4-yl)-1-oxo-2-buten-1-yl]amino}-7-cyclopentyloxyquinazoline;
4-[(3-chloro-4-fluorophenyl)amino]-6-{[4-((R)-6-methyl-2-oxomorpholin-4-yl-1-oxo-2-buten-1-yl]amino}-7-cyclopropylmethoxyquinazoline;
4-[(3-chloro-4-fluorophenyl)amino]-6-{[4-((R)-6-methyl-2-oxomorpholin-4-yl)-1-oxo-2-buten-1-yl]amino-}-7-[(S)-(tetrahydrofuran-3-yl)oxy]quinazoline;
4-[(3-chloro-4-fluorophenyl)amino]-6-{[4-((R)-2-methoxymethyl-6-oxomorpholin-4-yl)-1-oxo-2-buten-1-yl]amino}-7-cyclopropylmethoxyquinazoline;
4-[(3-chloro-4-fluorophenyl)amino]-6-[2-((S)-6-methyl-2-oxomorpholin-4-yl)ethoxy]-7-methoxyquinazoline;
4-[(3-chloro-4-fluorophenyl)amino]-6-({4-[N-(2-methoxyethyl)-N-methylamino]-1-oxo-2-buten-1-yl}amino)-7-cyclopropylmethoxyquinazoline;
4-[(3-chloro-4-fluorophenyl)amino]-6-{[4-(N,N-dimethylamino)-1-oxo-2-buten-1-yl]amino}-7-cyclopentyloxyquinazoline;
4-[(R)-(1-phenylethyl)amino]-6-{[4-(N,N-bis-(2-methoxyethyl)amino)-1-oxo-2-buten-1-yl]amino}-7-cyclopropylmethoxyquinazoline;
4-[(R)-(1-phenylethyl)amino]-6-({4-[N-(2-methoxyethyl)-N-ethylamino]-1-oxo-2-buten-1-yl}amino)-7-cyclopropylmethoxyquinazoline;
4-[(R)-(1-phenylethyl)amino]-6-({4-[N-(2-methoxyethyl)-N-methylamino]-1-oxo-2-buten-1-yl}amino)-7-cyclopropylmethoxyquinazoline;
4-[(R)-(1-phenylethyl)amino]-6-({4-[N-(tetrahydropyran-4-yl)-N-methylamino]-1-oxo-2-buten-1-yl}amino)-7-cyclopropylmethoxyquinazoline;
4-[(3-chloro-4-fluorophenyl)amino]-6-{[4-(N,N-dimethylamino)-1-oxo-2-buten-1-yl]amino}-7-((R)-tetrahydrofuran-3-yloxy)quinazoline;
4-[(3-chloro-4-fluorophenyl)amino]-6-{[4-(N,N-dimethylamino)-1-oxo-2-buten-1-yl]amino}-7-((S)-tetrahydrofuran-3-yloxy)quinazoline;
4-[(3-chloro-4-fluorophenyl)amino]-6-({4-[N-(2-methoxyethyl)-N-methylamino]-1-oxo-2-buten-1-yl}amino)-7-cyclopentyloxyquinazoline;
4-[(3-chloro-4-fluorophenyl)amino]-6-{[4-(N-cyclopropyl-N-methylamino)-1-oxo-2-buten-1-yl]amino}-7-cyclopentyloxyquinazoline;
4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]quinazoline;
4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]quinazoline;
4-[(3-chloro-4-fluorophenyl)amino]-6-[3-(morpholin-4-yl)propyloxy]-7-methoxyquinazoline;
4-[(3-ethynylphenyl)amino]-6,7-bis-(2-methoxyethoxy)quinazoline;
4-[(3-chloro-4-fluorophenyl)amino]-7-[3-(morpholin-4-yl)propyloxy]-6-[(vinylcarbonyl)amino]quinazoline;
4-[(R)-(1-phenylethyl)amino]-6-(4-hydroxyphenyl)-7H-pyrrolo[2,3-d]pyrimidine;
3-cyano-4-[(3-chloro-4-fluorophenyl)amino]-6-{[4-(N,N-dimethylamino)-1-oxo-2-buten-1-yl]amino}-7-ethoxyquinoline;
4-{[3-chloro-4-(3-fluorobenzyloxy)phenyl]amino}-6-(5-{[(2-methansulfonylethyl)amino]methyl}furan-2-yl)quinazoline;
Cetuximab;
Trastuzumab;
ABX-EGF; and
Mab ICR-62,
optionally in the form of a physiologically acceptable acid addition salt thereof.

6. The pharmaceutical composition according to claim 1, wherein the EGFR kinase inhibitor is selected from:
4-[(3-chloro-4-fluorophenyl)amino]-7-(2-{4-[(S)-(2-oxotetrahydrofuran-5-yl)-carbonyl]piperazin-1-yl}ethoxy)-6-[(vinylcarbonyl)amino]quinazoline;
4-[(3-chloro-4-fluorophenyl)amino]-7-[2-((S)-6-methyl-2-oxomorpholin-4-yl)ethoxy]-6-[(vinylcarbonyl)amino]quinazoline;
4-[(3-chloro-4-fluorophenyl)amino]-7-[4-((R)-6-methyl-2-oxomorpholin-4-yl)butyloxy]-6-[(vinylcarbonyl)amino]quinazoline;
4-[(3-chloro-4-fluorophenyl)amino]-7-[4-((S)-6-methyl-2-oxomorpholin-4-yl)butyloxy]-6-[(vinylcarbonyl)amino]quinazoline;
4-[(3-chloro-4-fluorophenyl)amino]-7-[4-(2,2-dimethyl-6-oxomorpholin-4-yl)butyloxy]-6-[(vinylcarbonyl)amino]quinazoline;
4-[(3-chloro-4-fluorophenyl)amino]-6-{[4-(morpholin-4-yl)-1-oxo-2-buten-1-yl]amino}-7-cyclopropylmethoxyquinazoline;
4-[(3-chloro-4-fluorophenyl)amino]-6-{[4-(N,N-diethylamino)-1-oxo-2-buten-1-yl]-amino}-7-cyclopropylmethoxyquinazoline;
4-[(3-chloro-4-fluorophenyl)amino]-6-{[4-(N,N-dimethylamino)-1-oxo-2-buten-1-yl]amino}-7-cyclopropylmethoxyquinazoline;
4-[(3-chloro-4-fluorophenyl)amino]-6-[(4-{N-[2-(ethoxycarbonyl)ethyl]-N-[(ethoxycarbonyl)methyl]amino}-1-oxo-2-buten-1-yl)amino]-7-cyclopropylmethoxyquinazoline;
4-[(R)-(1-phenylethyl)amino]-6-{[4-(morpholin-4-yl)-1-oxo-2-buten-1-yl]amino}-7-cyclopropylmethoxyquinazoline;
4-[(R)-(1-phenylethyl)amino]-6-{[4-(morpholin-4-yl)-1-oxo-2-buten-1-yl]amino}-7-cyclopentyloxyquinazoline;
4-[(3-chloro-4-fluorophenyl)amino]-6-{[4-((R)-6-methyl-2-oxomorpholin-4-yl)-1-oxo-2-buten-1-yl]amino}-7-cyclopropylmethoxyquinazoline;
4-[(3-chloro-4-fluorophenyl)amino]-6-({4-[bis-(2-methoxyethyl)amino]-1-oxo-2-buten-1-yl}amino)-7-cyclopropylmethoxyquinazoline;
4-[(3-chloro-4-fluorophenyl)amino]-6-{[4-((R)-6-methyl-2-oxomorpholin-4-yl)-1-oxo-2-buten-1-yl]amino}-7-[(S)-(tetrahydrofuran-3-yl)oxy]quinazoline;
4-[(3-chloro-4-fluorophenyl)amino]-6-{[4-((R)-2-methoxymethyl-6-oxomorpholin-4-yl)-1-oxo-2-buten-1-yl]amino}-7-cyclopropylmethoxyquinazoline;
4-[(3-chloro-4-fluorophenyl)amino]-6-[2-((S)-6-methyl-2-oxomorpholin-4-yl)ethoxy]-7-methoxyquinazoline;
4-[(3-chloro-4-fluorophenyl)amino]-6-({4-[N-(2-methoxyethyl)-N-methylamino]-1-oxo-2-buten-1-yl}amino)-7-cyclopropylmethoxyquinazoline;
4-[(3-chloro-4-fluorophenyl)amino]-6-{[4-(N,N-dimethylamino)-1-oxo-2-buten-1-yl]amino}-7-cyclopentyloxyquinazoline;
4-[(3-chloro-4-fluorophenyl)amino]-6-{[4-((S)-2-methoxymethyl-6-oxomorpholin-4-yl)-1-oxo-2-buten-1-yl]amino}-7-cyclopropylmethoxyquinazoline;
4-[(R)-(1-phenylethyl)amino]-6-{[4-(N,N-bis-(2-methoxyethyl)amino)-1-oxo-2-buten-1-yl]amino}-7-cyclopropylmethoxyquinazoline;
4-[(R)-(1-phenylethyl)amino]-6-({4-[N-(2-methoxyethyl)-N-ethylamino]-1-oxo-2-buten-1-yl}amino)-7-cyclopropylmethoxyquinazoline;
4-[(R)-(1-phenylethyl)amino]-6-({4-[N-(2-methoxyethyl)-N-methylamino]-1-oxo-2-buten-1-yl}amino)-7-cyclopropylmethoxyquinazoline;
4-[(R)-(1-phenylethyl)amino]-6-({4-[N-(tetrahydropyran-4-yl)-N-methylamino]-1-oxo-2-buten-1-yl}amino)-7-cyclopropylmethoxyquinazoline;
4-[(3-chloro-4-fluorophenyl)amino]-6-{[4-(N,N-dimethylamino)-1-oxo-2-buten-1-yl]amino}-7-((R)-tetrahydrofuran-3-yloxy)quinazoline;
4-[(3-chloro-4-fluorophenyl)amino]-6-{[4-(N,N-dimethylamino)-1-oxo-2-buten-1-yl]amino}-7-((S)-tetrahydrofuran-3-yloxy)quinazoline;
4-[(3-chloro-4-fluorophenyl)amino]-6-({4-[N-(2-methoxyethyl)-N-methylamino]-1-oxo-2-buten-1-yl}amino)-7-cyclopentyloxyquinazoline;
4-[(3-chloro-4-fluorophenyl)amino]-6-{[4-(N-cyclopropyl-N-methylamino)-1-oxo-2-buten-1-yl]amino}-7-cyclopentyloxyquinazoline;
4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]quinazoline;
4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]quinazoline;
4-[(3-chloro-4-fluorophenyl)amino]-6-[(4-dimethylaminocyclohexyl)amino]pyrimido[5,4-d]pyrimidine; and
4-[(3-chloro-4-fluorophenyl)amino]-6-[3-(morpholin-4-yl)propyloxy]-7-methoxyquinazoline, optionally in the form of a physiologically acceptable acid addition salt thereof.

7. The pharmaceutical composition according to claim 1, wherein the EGFR kinase inhibitor is selected from:
4-[(3-chloro-4-fluorophenyl)amino]-7-[4-((R)-6-methyl-2-oxomorpholin-4-yl)butyloxy]-6-[(vinylcarbonyl)amino]quinazoline;
4-[(3-chloro-4-fluorophenyl)amino]-7-[4-((S)-6-methyl-2-oxomorpholin-4-yl)butyloxy]-6-[(vinylcarbonyl)amino]quinazoline;
4-[(3-chloro-4-fluorophenyl)amino]-7-(2-{4-[(S)-(2-oxotetrahydrofuran-5-yl)-carbonyl]piperazin-1-yl}ethoxy)-6-[(vinylcarbonyl)amino]quinazoline;
4-[(3-chloro-4-fluorophenyl)amino]-7-[2-((S)-6-methyl-2-oxomorpholin-4-yl)ethoxy]-6-[(vinylcarbonyl)amino]quinazoline;
4-[(3-chloro-4-fluorophenyl)amino]-6-[(4-{N-[2-(ethoxycarbonyl)ethyl]-N-[(ethoxycarbonyl)methyl]amino}-1-oxo-2-buten-1-yl)amino]-7-cyclopropylmethoxyquinazoline;
4-[(R)-(1-phenylethyl)amino]-6-{[4-(morpholin-4-yl)-1-oxo-2-buten-1-yl]amino}-7-cyclopropylmethoxyquinazoline; and
4-[(3-chloro-4-fluorophenyl)amino]-6-[3-(morpholin-4-yl)propyloxy]-7-methoxyquinazoline,
optionally in the form of a physiologically acceptable acid addition salt thereof.

8. The pharmaceutical composition according to claim 1, wherein the weight ratios of the anticholinergic to the EGFR kinase inhibitor are in the range from 1:300 to 60:1.

9. The pharmaceutical composition according to claim 1, wherein the weight ratios of the anticholinergic to the EGFR kinase inhibitor are in the range from 1:200 to 30:1.

10. The pharmaceutical composition according to claim 1, wherein a single dose for administration corresponds to a dose of the active substance combination of the anticholinergic and the EGFR kinase inhibitor of 1000 μg to 100,000 μg.

11. The pharmaceutical composition according to claim 1, wherein a single dose for administration corresponds to a dose of the active substance combination of the anticholinergic and the EGFR kinase inhibitor of 1500 μg to 50,000 μg.

12. The pharmaceutical composition according to claim 1, wherein the pharmaceutical composition is in the form of a formulation suitable for inhalation.

13. The pharmaceutical composition according to claim 12, wherein the pharmaceutical composition is an inhalable powder, propellant-containing inhalable aerosol, or propellant-free inhalable solution or suspension.

14. The pharmaceutical composition according to claim 13, wherein the pharmaceutical composition is an inhalable powder comprising the anticholinergic and the EGFR kinase inhibitor in admixture with a suitable physiologically acceptable excipient selected from monosaccharides, disaccharides, oligo- and polysaccharides, polyalcohols, salts, or mixtures of these excipients with one another.

15. The inhalable powder according to claim 14, wherein the excipient has a maximum average particle size of up to 250 μm

16. The inhalable powder according to claim 15, wherein the excipient has a maximum average particle size of between 10 μm and 150 μm.

17. A capsule containing an inhalable powder according to claim 14.

18. The pharmaceutical composition according to claim 13, wherein the pharmaceutical composition is an inhalable powder consisting of the anticholinergic and the EGFR kinase inhibitor.

19. The pharmaceutical composition according to claim 13, wherein the pharmaceutical composition is a propellant-containing inhalable aerosol containing the anticholinergic and the EGFR kinase inhibitor in dissolved or dispersed form.

20. The propellant-containing inhalable aerosol according to claim 19, further comprising a propellant gas selected from a hydrocarbon or halohydrocarbon.

21. The propellant-containing inhalable aerosol according to claim 19, further comprising a propellant gas selected from n-propane, n-butane, isobutene, or chlorinated and/or fluorinated derivatives of methane, ethane, propane, butane, cyclopropane, or cyclobutane.

22. The propellant-containing inhalable aerosol according to claim 20, wherein the propellant gas is TG11, TG12, TG134a, TG227, or a mixture thereof.

23. The propellant-containing inhalable aerosol according to claim 20, wherein the propellant gas is TG134a, TG227, or a mixture thereof.

24. The propellant-containing inhalable aerosol according to claim 19, further comprising one or more other ingredients selected from cosolvents, stabilizers, surfactants, antioxidants, lubricants, and means for adjusting the pH.

25. The propellant-containing inhalable aerosol according to claim 19, wherein the propellant-containing inhalable aerosol contains up to 5 wt.-% of the anticholinergic and/or the EGFR kinase inhibitor active substances.

26. The pharmaceutical composition according to claim 13, wherein the pharmaceutical composition is a propellant-free inhalable solution or suspension which contains a solvent selected from water, ethanol, or a mixture of water and ethanol.

27. The inhalable solution or suspension according to claim 26, wherein the pH is 2 to 7.

28. The inhalable solution or suspension according to claim 26, wherein the pH is 2 to 5.

29. The inhalable solution or suspension according to claim 27, wherein the pH is adjusted by means of an acid selected from hydrochloric acid, hydrobromic acid, nitric acid, sulfuric acid, ascorbic acid, citric acid, malic acid, tartaric acid, maleic acid, succinic acid, fumaric acid, acetic acid, formic acid, and propionic acid, or mixtures thereof.

30. The inhalable solution or suspension according to claim 26, wherein the inhalable solution or suspension optionally contains other cosolvents and/or excipients.

31. The inhalable solution or suspension according to claim 30, further comprising a cosolvent selected from ingredients which contain hydroxyl groups or other polar groups.

32. The inhalable solution or suspension according to claim 30, further comprising a cosolvent selected from isopropyl alcohol, propyleneglycol, polyethyleneglycol, polypropylene glycol, glycol ether, glycerol, polyoxyethylene alcohols, and polyoxyethylene fatty acid esters.

33. The inhalable solution or suspension according to claim 30, further comprising an excipient selected from surfactants, stabilizers, complexing agents, antioxidants and/or preservatives, flavorings, pharmacologically acceptable salts, and/or vitamins.

34. The inhalable solution or suspension according to claim 33, further comprising a complexing agent selected from edetic acid or a salt of edetic acid.

35. The inhalable solution or suspension according to claim 33, further comprising containing sodium edentate.

36. The inhalable solution or suspension according to claim 33, further comprising an antioxidant selected from ascorbic acid, vitamin A, vitamin E, and tocopherols.

37. The inhalable solution or suspension according to claim 33, further comprising a preservative selected from cetyl pyridinium chloride, benzalkonium chloride, benzoic acid, and benzoates.

38. The inhalable solution or suspension according to claim 30, consisting of the anticholinergic, the EGFR kinase inhibitor, the solvent, benzalkonium chloride, and sodium edetate.

39. The inhalable solution or suspension according to claim 30, consisting of the anticholinergic, the EGFR kinase inhibitor, the solvent, and benzalkonium chloride.

40. The inhalable solution or suspension according to claim 26, wherein the inhalable solution or suspension is a concentrate or a sterile ready-to-use inhalable solution or suspension.

41. An inhaler containing a capsule according to claim 16.

42. An inhaler containing an inhalable solution according to claim 26.

43. A nebulizer containing an inhalable solution according to claim 40.

44. A method of treating an inflammatory or obstructive disease of the respiratory tract comprising administering to a patient in need of such treatment a therapeutically effective amount of the pharmaceutical composition according to claim 1.
